(19) United States
(12) Patent Application Publication
(10) Pub. No.: US 2020/0163657 A1
(43) Pub. Date: May 28, 2020

(54) POLYP TRAP

(71) Applicant: Designing Medical LLC, Palm Harbor, FL (US)

(72) Inventor: Robert Stuba, Palm Harbor, FL (US)

(21) Appl. No.: 16/630,202

(22) PCT Filed: Jul. 11, 2018
(86) PCT No.: PCT/US18/41668

§ 371 Date: Jan. 10, 2020

Related U.S. Application Data
(60) Provisional application No. 62/531,166, filed on Jul. 11, 2017.

(51) Int. Cl. A61B 10/02; A61B 10/04; A61M 1/00

(52) U.S. Cl. CPC A61B 10/0283; A61B 1/31; A61M 1/0056; A61B 10/04

(57) ABSTRACT

A polyp trap has an upper housing with a domed, top view window for viewing tissue retained by a disk seated in a lower housing which is held in place by the upper housing, when the upper housing is coupled to the lower housing. In one example, the upper housing holds the disk in place by engaging a post, which doubles as a handle for removing tissue from the trap with the disk and transferring the tissue to a storage container or bag. Mating posts in the lower housing may engage guide holes in the disk to further secure the disk in the trap.

### CROSS RELATED APPLICATIONS

This application is a 371 U.S. national phase application which claims priority to PCT/US2018/041668 filed Jul. 11, 2018 which claims priority to U.S. provisional application 62/531,166 filed Jul. 11, 2017, which is incorporated herein by reference in its entirety.

### FIELD OF THE INVENTION

The field relates to endoscopic biopsies and polyp removal and examination.

### BACKGROUND

Polyp traps are provided in some endoscopic procedures to separate out resected tissues, such as polyps, from other fluids. For example, U.S. Pat. No. 4,083,706 shows an early in-line filter that could be used as a polyp trap. International publication for PCT/DE97/00668 shows a more recent example of the same type of in-line filter element.

U.S. Pat. No. 4,685,472 shows a clear plastic, in-line trap. In U.S. Pat. Publ. 2011/0106029 a plastic trap has a flip top. U.S. Pat. No. 8,070,756 shows a trap integrated into an endoscopic biopsy system.

Some examples, such as U.S. Pat. No. 6,375,625, provide a bypass valve to direct fluids around the trap. U.S. Pat. D771,832 shows an ornamental design of a filter and cap element.

Some examples provide a plurality of baskets or a basket that can be removed from a side aperture during a procedure or both, such as U.S. Pat. Nos. 9,671,318; 7,244,236; and 9,220,485. The trend for polyp traps has been moving toward additional complexity in an attempt save time in the operating room, but adding complexity does not provid hoped for time savings. Instead, complexity adds costs, introduces complications and sometimes causes failures or complications during use.

### SUMMARY

A polyp trap comprises a domed upper housing with a view window, a porous separating disk with a handle, and a lower housing, wherein the disk is seated in the lower housing and is held in place by the upper housing, when the upper housing is coupled to the lower housing.

For example, the upper housing couples to the lower housing using interlocking flange members extending from the walls of the upper housing and the lower housing. For example, the upper housing holds the disk in place by engaging a post, a peripheral portion of the disk or both of these. In one example, the lower housing comprises mating posts that engage guide holes in the disk.

For example, a perforated region of the disk may be provided by a plurality of through holes, such that fluids may pass through the disk, while solid particles, such as polyps are trapped within the polyp trap by the disk.

Herein, a hole may be a blind hole, which does not extend through the thickness of the material, or a through hole, which does extend, completely through the thickness of the material, such that fluid is capable of passing through the through hole.

In one example, a polyp trap comprises an upper housing having a view window and an inlet; a separating disk; and a lower housing having an outlet, wherein the disk is seated in the lower housing, separating the outlet of the lower housing from the inlet of the upper housing, and

the disk has a perforated region provided by a plurality of through holes, such that fluids pass through the disk while solid particles larger than the through holes are trapped within the polyp trap by the disk, and the inlet and the view window of the upper housing are arranged such that the view window is disposed above the disk for viewing of any solid particles trapped in the polyp trap by the disk and the inlet does not obscure the view window's view of the disk.

For example, the disk may be held in place by the upper housing, when the upper housing is coupled to the lower housing. Alternatively, or in addition, the disk may be press fit or snap fit into or onto the lower housing or the upper housing. In one example, the upper housing couples to the lower housing using interlocking flange members extending from the walls of the upper housing and the lower housing. The upper housing may hold the disk in place by engaging a post, a peripheral portion of the disk or both of these, for example.

The lower housing may include mating posts and the disk may include guide holes such that the mating posts engage guide holes in the disk when the disk is seated in the lower housing, for example.

In one example, a plurality of through holes allow air, bodily fluids and smaller solid particles to pass freely through the disk while polyps are trapped in the poly trap. For example, the disk may include a handle extending upward from an upper face of the disk such that the disk may be removed from the lower housing by the handle, when the upper housing is removed from the lower housing. For example, the upper housing engages a top end of the handle when the upper housing is coupled to the lower housing such that the disk is held in place within the lower housing by contact between the upper housing and the handle. The lower housing may include mating posts and the disk may include guide holes such that the mating posts engage guide holes in the disk when the disk is seated in the lower housing and is held in place by contact of the upper housing with the handle.

In one example of using a polyp trap, according to these examples, the method comprises disposing the disk in the lower housing; fixing the upper housing onto the lower housing; coupling a suction line to the outlet of the lower housing; coupling a medical device to the inlet of the upper housing in fluid communication with the suction line; observing the disk through the view window; and removing polyps trapped in the polyp trap after the polyps are viewed through the view window. For example, the polyps may be disposed in a container for storage, transport or closer examination.

In one example, the inlet is arranged transversely to a direction of fluid flow through plurality of through holes of the disk such that the view of the disk through the view window on a domed upper surface of the upper housing is unimpeded by the inlet. In one example, the upper housing is made of an clear polymer and the lower housing and disk are opaque.

### BRIEF DESCRIPTION OF THE DRAWINGS

The following drawings are illustrative examples and do not further limit any claims that may eventually issue.

FIG. 1 is an example of an exploded view of a polyp trap.

FIG. 2 is a side view of an example of a disk.

FIG. 3 is a bottom view of an example of a disk.

FIG. 4 is a side view of an upper housing.

FIG. 5 is a cross sectional view of a lower housing.

When the same reference characters are used, these labels refer to similar parts in the examples illustrated in the drawings.

### DETAILED DESCRIPTION

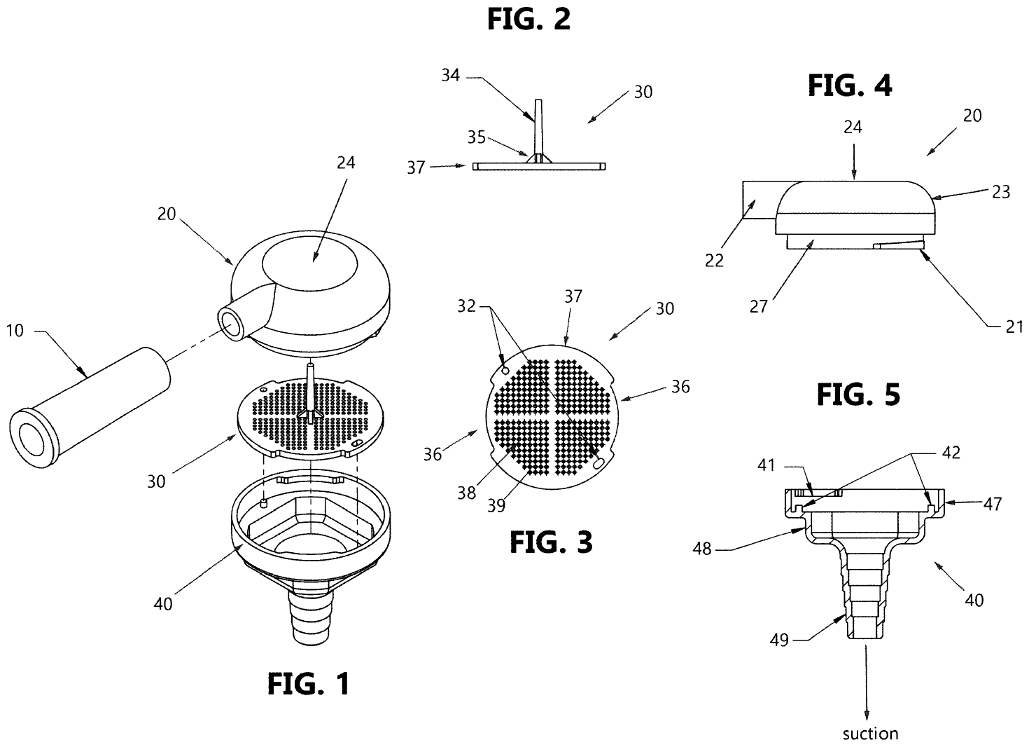

For example, FIG. 1 illustrates an exploded view of a polyp trap comprising a suction connector 10, a domed upper housing 20, a lower housing 40 and a perforated separating disk 30, separating the volume of the upper housing from a volume in the lower housing. In one example, the separating disk 30 is snap fit into place or press fit such that the separating disk 30 is fixed in the upper housing 20 or the lower housing 40.

For example, the upper housing may comprise a viewing window 24 disposed on a top portion of the upper housing. The window 24 may be made of a clear plastic, allowing a user to view any tissues retained within the polyp trap by the perforated disk.

The upper housing 20 comprises an inlet port 22 arranged extending from a side portion of the upper housing 20, such that the port 22 does not block the view through window 24. A domed portion 23 of the housing extends from the window 24. For example, the upper housing is made of a clear polymer substantially transparent to light and the view window 24 is integrally formed in the domed portion 23 of the upper housing 20.

For example, a rim flange 27 extends downwardly from the domed portion 23. The rim flange 27 may include one or more interlocking flanges 21 extending outwardly from the rim flange 27, for example. The rim flange of the upper housing 20 may be sized to fit into a rim 47 of the lower housing 40. The lower housing 40 may have one or more interlocking flanges 41 extending inwardly from the rim 47, which are arranged to matingly engage with corresponding interlocking flanges 21 of the upper housing 20.

A polygonal portion 48 of the lower housing 40 may be provided for gripping the lower housing 40, either by hand or with a tool. The polygonal portion 48 of the lower housing 40 makes it easier to tighten and loosen the upper housing 20 to/from the lower housing 40. In addition, the transition between the rim 47 and the polygonal portion 48 provides a step or shelf on which the disk 30 rests.

The window 24 allows viewing of any tissues that are retained by the porous, perforated separating disk 30. The disk 30 may comprise a plurality of small through holes 39 in one or more perforated regions 38 of the disk 30. In one example, the disk comprises a solid peripheral region 37 and a plurality of perforated regions 38. The peripheral region may comprise one or more cut-outs 36 for fitting over the interlocking flanges 41 that extend from the rim 47 of the lower housing 40.

The disk 30 may have one or more guide holes 32 formed in a surface of the disk 30. The guide holes 32 may be blind holes, as illustrated in the example of FIG. 3, or through holes, as illustrated in the exploded view in FIG. 1, for example. These guide holes 32 matingly fit onto posts 42 extending upwardly from a step or shelf formed by the wall of the lower housing 40, such as the step or shelf provided at the transition region between the rim 47 and the polygonal portion 48 of the lower housing 40, as best shown in FIG. 5, for example.

A connector outlet 49 may extend downwardly from the lower housing 40 and may be connected to a suction device, such as by a tube, for example.

The polyp trap may be used with an endoscopic system for taking a biopsy or for polyp removal, such as during a colonoscopy, for example. The inlet port 22 of the trap may be connected to an endoscopic device by a suction connector 10, for example, and the connector outlet 49 may be coupled to a suction device, providing suction for the endoscopic device introduced via an endoscope. Tissue and fluids may be suctioned through the inlet port 22, and tissue that is too large to fit through the perforations in the disk 30 may become trapped within the polyp trap.

The disk 30 may comprise a post 34 extending upwardly from a surface of the disk 30, as illustrated in FIG. 2, for example. The upper housing is easily removed from the lower housing, allowing the disk to be removed using the post 34 as a handle, for example. The tissue retained on the disk may be directed to a container or bag for collection and subsequent inspection. In one example, the window 24 is used to visually examine the tissue to determine when the tissue specimen should be removed from the trap.

The disk is may be reseated in the lower housing and may be held in place by the upper housing, when the upper housing is coupled to the lower housing. For example, the upper housing couples to the lower housing using interlocking flange members 21, 41, extending from the upper housing and the lower housing. For example, the upper housing holds the disk in place by engaging the post 34, a peripheral portion of the disk 37 or a combination of both of these. In one example, the lower housing comprises mating posts 42 that engage guide holes 32 in the disk. The guide holes 32 in the disk may be blind holes that extend only part-way through the thickness of the disk or through holes.

This detailed description provides examples including features and elements of the claims for the purpose of enabling a person having ordinary skill in the art to make and use the inventions recited in the claims. However, these examples are not intended to limit the scope of the claims, directly. Instead, the examples provide features and elements of the claims that, having been disclosed in these descriptions, claims and drawings, may be altered and combined in ways that are known in the art.

1. A polyp trap comprises:
an upper housing having a view window and an inlet;
a separating disk; and
a lower housing having an outlet,
wherein the disk is seated in the lower housing, separating the outlet of the lower housing from the inlet of the upper housing, and
the disk has a perforated region provided by a plurality of through holes, such that fluids pass through the disk while solid particles larger than the through holes are trapped within the polyp trap by the disk, and the inlet and the view window of the upper housing are arranged such that the view window is disposed above the disk for viewing of any solid particles trapped in the polyp trap by the disk and the inlet does not obscure the view window's view of the disk.

2. The polyp trap of claim 1, wherein the disk is held in place by the upper housing, when the upper housing is coupled to the lower housing.

3. The polyp trap of claim 1, wherein the upper housing couples to the lower housing using interlocking flange members extending from the walls of the upper housing and the lower housing.

4. The polyp trap of claim 1, wherein the upper housing holds the disk in place by engaging a post, a peripheral portion of the disk or both of these.

5. The polyp trap of claim 1, wherein the lower housing includes mating posts and the disk includes guide holes, and the mating posts engage guide holes in the disk when the disk is seated in the lower housing.

6. The polyp trap of claim 1, wherein the disk is fixed in the lower housing.

7. The polyp trap of claim 1, wherein the plurality of through holes allow air, bodily fluids and smaller solid particles to pass freely through the disk while polyps are trapped in the poly trap.

8. The polyp trap of claim 1, wherein the upper housing is made of a clear polymer such that the view window is integrally formed in the upper housing.

9. The polyp trap of claim 1, wherein disk includes a handle extending upward from an upper face of the disk such that the disk may be removed from the lower housing by the handle, when the upper housing is removed from the lower housing.

10. The polyp trap of claim 9, wherein the upper housing engages a top end of the handle when the upper housing is coupled to the lower housing such that the disk is held in place within the lower housing.

11. The polyp trap of claim 10, wherein the lower housing includes mating posts and the disk includes guide holes, and the mating posts engage guide holes in the disk when the disk is seated in the lower housing.

12. The polyp trap of claim 11, wherein the plurality of through holes allow air, bodily fluids and smaller solid particles to pass freely through the disk while polyps are trapped in the poly trap.

13. The polyp trap of claim 1, wherein the inlet is arranged transversely to a direction of fluid flow through plurality of through holes of the disk such that the view of the disk through the view window of the upper housing is unimpeded by the inlet.

14. A method of using the polyp trap of claim 1, comprising:
disposing the disk in the lower housing;
fixing the upper housing onto the lower housing;
coupling a suction line to the outlet of the lower housing;
coupling a medical device to the inlet of the upper housing in fluid communication with the suction line;
observing the disk through the view window; and
removing polyps trapped in the polyp trap after the polyps are viewed through the view window.
